(19) United States
(12) Patent Application Publication
(10) Pub. No.: US 2012/0207730 A1
(43) Pub. Date: Aug. 16, 2012

(54) Methods and Compositions for Treating Viral Hemorrhagic Fever

(75) Inventors: Betty A. Wu-Hsieh, Arcadia, CA (US); Yu-Ting Yen, Taipei City (TW)

(73) Assignee: NATIONAL TAIWAN UNIVERSITY, Taipei (TW)

(21) Appl. No.: 13/459,358

(22) Filed: Apr. 30, 2012

Related U.S. Application Data
(62) Division of application No. 12/546,992, filed on Aug. 25, 2009.
(60) Provisional application No. 61/194,272, filed on Sep. 26, 2008.

(51) Int. Cl. A61K 31/12; A61K 31/255; A61P 7/04; A61K 31/343; A61K 31/381; A61K 38/43; A61K 31/225; A61K 31/366

(52) U.S. Cl. 424/94.1; 514/678; 514/547; 514/517; 514/460; 514/469; 514/443

(57) ABSTRACT

Disclosed herein are methods and compositions for treating viral hemorrhagic fever in a subject by use of at least one inhibitor or suppressor of oxidase, inducible nitric oxide synthase (iNOS) and apoptosis pathway.

### RELATED APPLICATIONS

The present application is a divisional of U.S. application Ser. No. 12/546,992, filed on Aug.25, 2009, which was based on, and claims priority from, U.S. Patent Application Ser. No. 61/194,272, filed Sep. 26, 2008, the disclosure of which is hereby incorporated by reference herein in its entirely.

### BACKGROUND

1. Field of Invention

The present invention in general relates to methods and compositions for treating viral hemorrhagic fever in a subject. More particularly, the present invention relates to methods and compositions for treating dengue virus induced hemorrhage in a subject by use of at least one inhibitor or suppressor of oxidase, inducible nitric oxide synthase (iNOS) or apoptosis pathway to reduce hemorrhage development.

2. Description of Related Art

Dengue virus (DENV), a member of the family Flaviviridae, is a mosquito-borne virus with four serotypes (DENV-1, -2, -3, and -4). The four serotypes cause dengue epidemics in South East Asia, Central America, and the Pacific region. Clinical illnesses in humans range from a flu-like disease of dengue fever (DF) to a fulminating illness of hemorrhagic fever (DHF), which can progress to dengue shock syndrome (DSS) and death. Severe hemorrhage with low platelet counts, plasma leakage and pleural or other effusions, and increased vascular permeability are characteristics of DHF/DSS. Thus, it is apparent that vascular damage plays a key role in the pathophysiology of severe dengue disease.

The endothelium forms the primary barrier of the circulatory system. Vascular damage in DHF patients is evidenced by the presence of circulating endothelial cells (CECs) in the peripheral blood. A recent report revealed the presence of apoptotic microvascular endothelial cells in the pulmonary and intestinal tissue samples from fatal cases of DSS, suggesting that endothelial cell apoptosis is related to vascular damage. (See, Limonta, D. et al., “Apoptosis in tissues from fatal dengue shock syndrome”, J Clin Virol 40:50-4, 2007) Both host and viral factors are known to contribute to the alteration of vascular endothelium in dengue disease. In examining biopsy and autopsy tissue specimens from patients who died of DHF/DSS, Jessie et al. detected dengue viral antigens in the endothelial cells in the lung and liver tissues, suggesting that dengue virus might directly interact with endothelial cells in humans. (See, Jessie, K. et al., “Localization of dengue virus in naturally infected human tissues, by immunohistochemistry and in situ hybridization”. J Infect Dis 189:1411-8, 2004) In previous research conducted by the inventor of this application, it is observed in the mouse model that there is endothelium damage and apoptotic endothelial cell death in hemorrhage tissues, which corresponds to the observation made in human dengue hemorrhage. (See, Chen, H. C. et al., “Both virus and tumor necrosis factor alpha are critical for endothelium damage in a mouse model of dengue virus-induced hemorrhage”, J Virol 81:5518-26, 2007) These studies together suggest the importance of endothelial cell apoptosis in vascular damage.

The critical role of TNF-α in the cause of severe dengue disease has long been recognized. Bethell et al. observed a positive relationship between high levels of TNFR and the severity of DHF. (See, Bethell D B, et al., “Pathophysiologic and prognostic role of cytokines in dengue hemorrhagic fever”, J Infect Dis. March; 177(3):778-82, 1998.) Single nucleotide polymorphism analysis also identified TNF-α polymorphism at TNF-308A allele to be a possible risk factor for hemorrhagic manifestations in dengue patients. (See, Fernandez-Mestre, M. T. et al., “TNF-α-308A allele, a possible severity risk factor of hemorrhagic manifestation in dengue fever patients”, Tissue Antigens 64:469-72, 2004.) In previous research conducted by the inventor of this application, the direct causal relationship between TNF-α and dengue hemorrhage was established in the mouse model, in that TNF-α deficiency greatly diminishes hemorrhage development (See, Chen et al, 2007, supra). However, how TNF-α together with dengue virus cause endothelium damage remains a question to be addressed.

There is therefore a great need of developing a treatment for use in treating or preventing dengue virus induced hemorrhage.

### SUMMARY

Aspects of this invention relate to methods and compositions for treating viral hemorrhagic fever in a subject.

Accordingly, it is an aspect of this invention to provide a pharmaceutical composition for treating viral hemorrhagic fever in a subject, comprising at least one inhibitor selected from the group consisting of an oxidase inhibitor, an inducible nitric oxide synthase (iNOS) inhibitor and an inhibitor of apoptosis pathway; and a pharmaceutically acceptable excipient. In one embodiment, the pharmaceutical composition may further comprise a tumor necrosis factor alpha (TNF-α) inhibitor. In one embodiment, the viral hemorrhagic fever is caused by dengue virus infection.

It is a further aspect of this invention to provide a method of treating viral hemorrhagic fever in a subject. The method comprises administering to the subject a therapeutically effective amount of a pharmaceutical composition comprising at least one inhibitor selected from the group consisting of an oxidase inhibitor, an inducible nitric oxide synthase (iNOS) inhibitor and an inhibitor of apoptosis pathway to reduce the hemorrhage development in the subject. The pharmaceutical composition may further comprise a TNF-α inhibitor.

In one embodiment of the invention, the oxidase inhibitor includes inhibitor of ceramide-NADPH pathway, such as apocynin (i.e., 4-hydroxy-3-methoxyacetophenone) or fumonisin B1. The iNOS inhibitor is N-acetyl cysteine (NAC) or N^{G}-nitro-L-arginine methyl ester (L-NAME). The inhibitor of apoptosis pathway comprises a caspase inhibitor, such as Boc-D-FMK or Z-Asp-CH₂-DCB. The TNF-α inhibitor is a monoclonal antibody of a TNF-α receptor or a nature compound.

These and other features, aspects, and advantages of the present invention will become better understood with reference to the following description and appended claims. It is to be understood that both the foregoing general description and the following detailed description are by examples, and are intended to provide further explanation of the invention as claimed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention can be more fully understood by reading the following detailed description of the embodiment, with reference made to the accompanying drawings as follows:

FIGS. 1A to 1C illustrate dengue viral antigen (FIGS. 1A and 1B) can be detected in CD31-positive vascular endothelial cells and viral nucleic acids (FIG. 1C) in vascular endothelium after infection in accordance with one embodiment of this disclosure;

FIGS. 2A to 2C illustrate hemorrhage induced by infiltrating macrophages in the vicinity of endothelium (2A), TNF-α production (2B) and endothelial cell death (2C), respectively in accordance with one embodiment of this disclosure;

FIG. 3 illustrates the reduction of hemorrhage development by caspase inhibitor in DENV-infected mice in accordance with one embodiment of this disclosure;

FIGS. 4A to 4C illustrate the enumeration of circulating endothelial cells in accordance with one embodiment of this disclosure;

FIGS. 5A and 5B respectively illustrate the expression of iNOS (5A) and nitrotyrosine (5B) in the endothelium of hemorrhage tissues in accordance with one embodiment of this disclosure;

FIGS. 6A to 6E illustrate the productivity of DENV in the infection of endothelial cells and the induction of apoptosis in accordance with one embodiment of this disclosure;

FIGS. 7A to 7F illustrate the induced iNOS expression and RNS and ROS production in dengue virus infected endothelial cells (FIGS. 7A to 7D) and both RNS and ROS are key to dengue virus-induced apoptosis (FIGS. 7E and 7F) in accordance with one embodiment of this disclosure;

FIGS. 8A to 8D illustrate the effect of TNF-α on DENV-induced endothelial cell apoptosis (FIGS. 8A to 8C) and the effect can be reversed by RNS and ROS inhibitors and apoptosis inhibitors (FIG. 8D) in accordance with one embodiment of this disclosure;

FIG. 9 illustrates the contribution of endothelial cell apoptosis on permeability change in accordance with one embodiment of this disclosure; and

FIG. 10 illustrates that both RNS and ROS contribute to hemorrhage development in DENV-infected mice in accordance with one embodiment of this disclosure.

### DETAILED DESCRIPTION

While the present invention may be embodied in many different forms, several specific embodiments are discussed herein with the understanding that the present disclosure is to be considered only as an exemplification of the principles of the invention, and it is not intended to limit the invention to the embodiments illustrated.

In the following disclosure, methods and compositions for treating viral hemorrhagic fever in a subject are described. Hemorrhagic fever viruses are RNA viruses such as those of Filoviridae, Arenaviridae, Bunyaviridae or Flaviviridae family, include but are not limited to ebola viruses, marburg viruses, flexal viruses, guanarito viruses, jumin viruses, lassa fever viruses, machupo viruses, sabia viruses, crimea-congo hemorrhagic fever viruses, rift valley fever viruses, hantaan viruses, dengue viruses, kyasanur forest disease viruses, omsk hemorrhagic fever viruses, and yellow fever viruses. In one aspect of the invention, the Hemorrhagic fever viruses are dengue viruses.

Inventors of this application unexpectedly identify that dengue viruses-induced endothelial cell production of reactive nitrogen and oxygen species (RNS and ROS) and apoptotic cell death, were greatly enhanced by tumor necrosis factor alpha (TNF-α). Therefore, if an agent has the efficacy of inhibiting cell death or the production of TNF-α or ROS and RNS, the agent can be used to treat dengue virus-induced hemorrhage. The use of the identified agents in the treatment of dengue virus-induced hemorrhage has been demonstrated in the examples herein. The agents include but are not limited to inhibitors or suppressors of oxidase, inducible nitric oxide synthase (iNOS) or apoptosis pathway. A TNF-α inhibitor may be administered before, at the same time or after administering the identified agents to a subject in need of such treatment to suppress the hemorrhagic development.

It is therefore an aspect of the invention to provide a pharmaceutical composition for treating viral hemorrhagic fever in a subject. The composition comprises at least one inhibitor selected from the group consisting of an oxidase inhibitor, an inducible nitric oxide synthase (iNOS) inhibitor and an inhibitor of apoptosis pathway; and a pharmaceutically acceptable excipient. In one embodiment, the pharmaceutical composition of this invention may further comprise a TNF-α inhibitor.

In one example, the oxidase inhibitor is an inhibitor of ceramide-NADPH oxidase, such as apocynin (i.e., 4-hydroxy-3-methoxyacetophenone), or an inhibitor of ceramide synthesis, such as fumonisin B1. Examples of other types of NADPH oxidase inhibitors that may be useful include, but are not limited to, isoprenylation inhibitors such as lovastatin and compactin (see U.S. Pat. No. 5,224,916), benzofuranyl- and benzothienyl thioalkane carboxylates (see EP 551,662), and cytochrome b₅₅₈ fragments and their analogs (see WO 91/17763).

In some examples, the iNOS inhibitors or suppressors may be inhibitors of Ras/Raf/MAP kinase pathway. In certain embodiments, the iNOS inhibitors or suppressors may be inhibitors of NF-kB, such as an inhibitor of NF-kB activation, and/or a suppressor of its induction. The inhibitor of NF-kB includes, but is not limited to, lovastatin, phenylacetate, metastatin, 4-phenylbutyrate, 5-aminoimmidazole-4-carboxamide ribonucleoside (AICAR) and salts, analogs, or derivatives thereof. In some embodiments, the iNOS inhibitor or suppressor may be an inhibitor of mevalonate synthesis. In certain embodiments, the inhibitor of mevalonate synthesis may be an inhibitor of the farnasylation of a protein. In certain embodiments, the inhibitor of mevalonate synthesis may be an inhibitor of HMG-CoA reductase and/or suppressor of its induction, including, but is not limited to, lovastatin or AICAR and salts, analogs or derivatives thereof. In certain embodiments, the inhibitor of HMG-CoA reductase is a simulator of AMP-activated protein kinase, including, but is not limited to, AICAR and salts, analogs or derivatives thereof. In certain embodiments, the iNOS inhibitors or suppressors may be a stimulator of AMP-activated protein kinase. In certain embodiments, the iNOS inhibitors or suppressors may be an inhibitor of mevalonate pyrophosphate decarboxylase and/or suppressor of its induction, including, but is not limited to, phenylacetic acid, 4-phenylbutyrate and salts, analogs or derivatives thereof.

In other embodiments, the iNOS inhibitors or suppressors may be an antioxidant. In some embodiments, the antioxidant may be, but is not limited to, N-acetyl cysteine, N^{G}-nitro-L-arginine methyl ester, and salts, analogs or derivatives thereof.

In certain embodiments, the iNOS inhibitors or suppressors may be an enhancer of intracellular AMP, inhibitor of the Ras/Raf/MAP kinase pathway, inhibitor of NF-kB, NF-kB activation and/or suppressor of NF-kB induction. In one embodiment, the inhibitor of the Ras/Raf/MAP kinase pathway includes, but is not limited to, AICAR and salts, analogs or derivatives thereof. The enhancer of intracellular cAMP may be an inhibitor of cAMP phosphodiesterase and/or suppressor of its induction. In some aspects of the invention, the inhibitor of cAMP phosphodiesterase may be, but is not limited to, rolipram and salts, analogs or derivatives thereof. In certain other aspects of the invention, the iNOS inhibitors or suppressors is cAMP and salts, analogs or derivatives thereof. Derivatives of cAMP include, but are not limited to, 8-bromo-cAMP or (S)-cAMP. In other aspects of the invention, the enhancer of intracellular cAMP may be, but is not limited to, forskolin, rolipram, 8-bromo-cAMP, theophylline, papaverine, cAMP and salts, analogs or derivatives thereof. In certain embodiments, the iNOS inhibitors or suppressors may be an enhancer of protein kinase A. In other aspects of the invention, the enhancer of protein kinase A may include, but is not limited to, forskolin, rolipram, 8-bromo-cAMP, theophylline, papaverine, cAMP and salts, analogs or derivatives thereof.

In one embodiment of the invention, the iNOS inhibitor or suppressor is selected from the group consisting of lovastatin, mevastatin, forskolin, rolipram, phenylacetate, N-acetyl cysteine, N^{G}-nitro-L-arginine methyl ester, pyrolidine dithiocarbamate, 4-phenylbutyrate, 5-amminoimmidazole-4-carboxamide ribonucleoside (AICAR), theophyllin, papaverine, cAMP, 8-bromo-cAMP, (S)-cAMP, and salts, analogs or derivatives thereof.

The inhibitors of apoptosis pathway are compounds that suppress or inhibit enzymes, such as caspases, in the signaling pathway for programmed cell death. Caspases are a family of cysteine protease enzymes that are key mediators in apoptosis pathway, and are classified into 3 groups depending on the amino acid sequence that is preferred or primarily recognized. The group of caspases, which includes caspases 1, 4 and 5, has been known to prefer hydrophobic aromatic amino acids at position 4 on N-terminal side of the cleavage site. Another group that includes caspases 2, 3 and 7, recognize aspartyl residues at both positions 1 and 4 on the N-terminal side of the cleavage site, and preferably a sequence of Asp-Glu-X-Asp. A third group, which includes caspases 6, 8, 9 and 10 tolerate many amino acids in the primary recognition sequence, but seems to prefer residues with branched, aliphatic side chain such as valine and leucine at position 4. Knowledge of the four amino acid sequence primarily recognized by the caspases has been used to design caspase inhibitors. Reversible and irreversible tetrapeptide inhibitors based on recognition sequence capable of binding to the caspase cysteine sulfhydryl group have been prepared (See WO 99/47154, WO 99/18781, and WO 00/61542). To increase cell permeability of the caspase inhibitors, aspartic acid residues are esterified (OMe). In selected case, a long peptide corresponding to the hydrophobic region of Kaposi fibroblast growth factor is added to the recognition sequence to increase cell permeability. Some caspase inhibitors are biotinylated for easy isolation, identification and characterization of different caspases. Examples of suitable caspases inhibitors may be obtained from CALBIOCHEM (San Diego, Calif., USA), which include, but are not limited to, Z-VAD-FMK, Biotin-X-VAD-FMK, Ac-VAD-CHO,

Boc-D-FMK, Ac-YVAD-CHO, Ac-YVAD-CMK, Biotin-YVAD-CMK, Z-Asp-CH₂-DCB, Z-YVAD-FMK, Z-VDVAD-FMK, Biotin-DEVD-CHO, Ac-DEVD-CHO, Z-DEVD-FMK, Ac-DEVD-CMK, Biotin-X-DEVD-FMK, Z-IETD-FMK and Z-WHED-FMK; where Ac represents acetyl, Boc represents tert-butyloxycarbonyl, CMK represents chloromethylketone, FMK represents fluoro-methyl ketone, X represents a linker, Z represents benzyloxycarbonyl, and Biotin represents biotin group. In one example of the invention, the caspase inhibitor is Boc-D-FMK or Z-Asp-CH₂-DCB.

In another aspect of the invention, the pharmaceutical composition prepared in accordance with this disclosure further comprises a TNF-α inhibitor, which may be a monoclonal antibody of a TNF-α receptor, includes but is not limited to, infliximab (trade name REMICADE®), adalimumab (trade name HUMIRA®), golimumab (also known as CNTO 148), certolizumab pegol (trade name CIMZIA®) and afelimomab (also known as Fab 2 or MAK 195F); or a TNF-α receptor fusion protein such as etanercept (trade name ENBREL®); or a nature compound including, but not limited to curcumin and catechins.

When the terms as used herein are referred to, the terms are defined as follows. Any undefined terms have the definitions recognized by persons skilled in the art.

A “salt” is understood here in certain embodiments to mean a compound formed by the interaction of an acid and a base, the hydrogen atoms of the acid being replaced by the positive ion of the base. Salts, within the scope of this invention, include both the organic and inorganic types and include, but are not limited to, the salts formed with ammonia, organic amines, alkali metal hydroxides, alkali metal carbonates, alkali metal bicarbonates, alkali metal hydrides, alkali metal alkoxides, alkali earth metal hydroxides, alkali earth metal carbonates, alkali earth metal bicarbonates, alkali earth metal hydrides, alkali earth metal alkoxides. Representative examples of bases that form such base salts include ammonia, primary amines such as n-propylamine, n-butylamine, aniline, cyclohexylamine, benzylamine, ethanolamine and glucamine; secondary amines such as diethylamine, diethanolamine, N-methylglucamine, N-methylaniline, morpholine, pyrrolidine and piperidine; tertiary amines such as triethylamine, triethanolamine, N,N-dimethylaniline, N-ethylpeperidine and N-methylmorpholine; hydroxides such as sodium hydroxides; alkoxides such as sodium ethoxide and potassium methoxide; hydrides such as calcium hydride and sodium hydride; and carbonates such as potassium carbonate and sodium carbonate. According to some embodiment of the present invention, the salts are those of sodium, potassium, ammonium, ethanolamine, diethanolamine and triethanolamine. Particularly are the sodium salts.

As used herein, “derivatives” refers to chemically modified inhibitors or suppressors that still retain the desired inhibiting or suppressing property of the unmodified inhibitors or suppressors. Such derivatives may be prepared by any method known to those of skill in the art.

As used herein, “analogs” include structural equivalents or mimetics of a compound.

The term “treat”, “treating” or “treatment” refers to the administration of therapy to a subject, particularly a mammal, more particularly a human, who already manifests at least one symptom of dengue disease. Such a subject includes an individual who is diagnosed as having dengue disease.

The term “pharmaceutically acceptable excipient” as used herein generally refers to organic or inorganic materials, which cannot react with active ingredients. The excipients include but are not limited to sugars, such as lactose, glucose and sucrose; starches such as corn starch and potato starch; cellulose and its derivatives such as sodium carboxymethylcellulose, ethylcellulose, and cellulose acetate; powered tragancanth; malt; gelatin; talc; stearic acids; magnesium stearate; calcium sulfate; vegetable oils, such as peanut oil, cotton seed oil, seasame oil, olive oil, corn oil and oil of theobroma; polyols such as propylene glycol, glycerine, sorbitol, mannitol, and polyethylene glycol; agar; alginic acids; pyrogen-free water; isotonic saline; and phosphate buffer solution; skim milk powder; as well as other non-toxic compatible substances used in pharmaceutical formulations. Wetting agents and lubricants such as sodium lauryl sulfate, as well as coloring agents, flavoring agents, lubricants, carriers, tabletting agent, stabilizers, antioxidants and preservatives, can also be present.

The term “therapeutically effective amount” as used herein generally refers to an amount of an agent, for example the amount of a compound as an active ingredient, that is sufficient to effect treatment as defined herein when administered to a subject in need of such treatment. A therapeutically effective amount of a compound, salt, analog, or derivative of the present invention will depend on a number of factors including, for example, the age and weight of the subject, the precise condition requiring treatment and its severity, the nature of the formulation, and the route of administration, and will ultimately be at the discretion of the attendant physician. However, an effective amount of the compound of the present invention for the treatment of symptoms of hemorrhagic fever, particularly dengue virus-induced hemorrhagic fever, will generally be in the range of about 60 to about 80 mg/kg body weight of recipient (mammal) per day and more particularly about 70 mg/kg body weight of recipient (mammal) per day. Thus, for a 70 kg adult subject, the actual amount per day would be about 4900 mg, and this amount may be given in a single dose per day or more usually in a number (such as 2, 3, 4, 5 or 6) of sub-doses per day such that total daily dose is the same.

In administering the inhibitor or suppressor of oxidase, inducible nitric oxide synthase (iNOS) or apoptosis pathway to a subject, preferably a human, the inhibitor or suppressor is formulated into a pharmaceutically acceptable vehicle. The inhibitor or suppressor of oxidase, inducible nitric oxide synthase (iNOS) or apoptosis pathway may be administered to a subject in a dose therapeutic to treat hemorrhagic conditions associated with dengue virus infection, where there is an advantage in inhibiting or suppressing the induction of oxidase, inducible nitric oxide synthase (iNOS) or apoptosis pathway.

A “subject”, as used herein, may be an animal. Preferred animals are mammals, including but are not limited to humans, pigs, cats, dogs, rodents or cattle including but are not limited to sheep, goats and cows. Preferred subject is human.

It must be noted that as used herein and in the appended claims, the singular forms “a”, “an”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice of the present invention, exemplary methods and materials are described for illustrative purposes.

Reference will now be made in detail to the present embodiments of the invention, examples of which are illustrated in the accompanying drawings.

The following materials and method and Examples are provided to illustrate certain aspects of the present invention and to aid those of skilled in the art in practicing this invention. In the Examples presented, Student's t test was used to compare the difference between groups. Results are reported as mean±standard deviation. These Examples are in no way to be considered to limit the scope of the invention in any manner.

### EXAMPLES

### Materials and Methods

### Mice and DENV Infection

C57BL/6, iNOS−/− (Nos2tm1Lau3/J) mice were originally obtained from the Jackson Laboratory (Bar Harbor, Me.) and bred at the Laboratory Animal Center of National Taiwan University College of Medicine. Male p47^{phox-/-} (B6(Cg)-Ncf1m1J6 /J) mice were obtained from the Jackson Laboratory and housed in micro-isolator cages in pathogen-free and environmentally controlled conditions at the Laboratory Animal Center of National Cheng Kung University.

Dengue virus type 2 strain 16681 was used throughout this study. Generally, the DENV infection was conducted as follows. Mice were inoculated with type 2 DENV strain 16681 (in 0.4 ml) intradermally at 4 sites (as 4 corners of a rectangle) on the upper back as previously described in Chen et al, 2007, supra. Mice given PBS or UV-inactivated DENV through the same route were used as controls. At 3 days after infection, mice were sacrificed and all tissues were examined to observe hemorrhage development.

H & E staining was used to stain the tissue. Tissues were fixed in 4% neutral formalin solution, embedded in paraffin, and sectioned at 3 μm thickness. After deparaffinization and rehydration, the sections were stained with hematoxylin and eosin. The sections were dehydrated before mounting.

### Immunofluorescence Staining of Cryosections

To observe the expression of some specific proteins/nucleic acids, subcutaneous tissues collected from mice at given time after the intradermal inoculation were treated as follows.

Tissues were snapped frozen in liquid nitrogen and the cryosections were fixed in acetone for 5 min then blocked by treating with PBS containing 5% goat serum at room temperature for 20 min.

Before staining, cryosections were fixed in 4% paraformaldehyde for 10 min at room temperature before 1-hour treatment with MOM mouse IgG blocking reagent (Vector, Burlingame, Calif.). Cryosections were then stained with specific stains described in relevant Examples provided hereinafter. RT-PCR amplification and detection of TNF-α and iNOS mRNA

RT-PCR was conducted to amplify the TNF-α and iNOS mRNA in tissues. Total RNA was extracted from skin tissues using Trizol reagent (Invitrogen, Carlbad, Calif.) according to the method described in Chen et al, 2007, supra. The amplification condition was 90 for 3 min, followed by 35 cycles of 94 for 40 sec, 69 for 1 min, 72 for 1 min, then 72 for 10 min, and the reaction was stopped at 4 . Housekeeping gene HPRT was used as a control.

The PCR products were subjected to electrophoresis on a 2% agarose gel. The sequence of iNOS primer set was 5′-TGGGAATGGAGACTGTCCCAG-3′ (SEQ ID NO: 1) and 5′-GGGATCTGAATGTGATGTTTG -3′ (SEQ ID NO: 2) (see, Maffei, C. M. et al., “Cytokine and inducible nitric oxide synthase mRNA expression during experimental murine cryptococcal meningoencephalitis”, Infect Immun 72:2338-49, 2004.) and that for TNF-α primers was 5′-ATCCGCGACCTCGCCCTG-3′ (SEQ ID NO: 3) and 5′-ACCGCCTGGAGTTCTGGAA-3′ (SEQ ID NO: 4). The sequence for HPRT primer set was 5′- GTTGGATACAGGCCAGACTTTGTTG-3′ (SEQ ID NO: 5) and 5′- GAGGGTAGGCTGGCCTATGGCT-3′ (SEQ ID NO: 6) (See, Chen et al, 2007, supra).

### Flow Cytometry Analysis

Flow cytometric analysis was used to enumerate the circulating endothelial cells (CECs). Peripheral blood was collected from mice and the red blood cells were lysed in BD FACSTM Lysing Solution (BD Bioscience, San Jose, Calif.) and stained with FITC-conjugated rat anti-mouse CD31 (clone PECAM-1), APC-conjugated rat anti-mouse VEGFR-2 (clone 89B3A5), and PE/Cy7-conjugated rat anti-mouse CD45 (clone 30-F11) (all from BioLegend, San Diego, Calif.) antibodies. Cells were acquired by a FACSCanto flow cytometer (BD Biosciences) and analyzed by BDFACSDiva flow cytometric analysis software (BD Biosciences). Platelets and cellular debris were excluded.

Infection of MBECs and HUVECs with DENVs and Determination of Cell Viability and Transendothelial Permeability

Both mouse microvascular endothelial cells (MBEC) and human umbilical vascular endothelial cells (HUVEC) were infected with DENV. HUVECs were prepared as previously described by Jaffe, E. A. et al. in “Culture of human endothelial cells derived from umbilical veins—Identification by morphologic and immunologic criteria”, J Clin Invest 52:2745-56, 1973. The method of isolating mouse brain microvascular endothelial cells (MBEC) was adopted from a published protocol (Wu, Z., et al., “A simple method for isolation and characterization of mouse brain microvascular endothelial cells”, J Neurosci Methods 130:53-63, 2003).

HUVEC and MBEC at passages 3-5 and 2-4, respectively, were used in experiments. About 5×10⁴ endothelial cells (HUVEC or MBEC) were seeded onto 13 mm round coverslips (Polylab, Strasbourg, France) pre-coated with 1% gelatin. After overnight incubation, the cells were cultured in medium containing 2% inactivated fetal bovine serum before different titers of live or UV-inactivated dengue virus was added. The cells were incubated at 37° C. for 2 h with gentle shaking every 10 min. After wash, the culture supernatants were harvested for NOx determination and plaque assay and infected endothelial cells for viral Ag determination.

The viability of HUVEC after DENV infection was quantified by their ability to reduce 3-[4,5-dimethylthiazol]-2,5-diphenyltetrazolium bromide (MTT) to formazan precipitate in quadruplicate wells. MTT (Sigma-Aldrich) at a final concentration of 5 mg/mL was added to each well at 3 h before termination of the experiment. The formazan dye was dissolved by incubation in DMSO (Merck, Berlin, Germany) and its concentration was determined spectrophotometrically at an absorbance wavelength of 560 nm. The percentage of cell death was calculated based on the OD560 reading by the following formula: Percentage of cell death=100×(OD560 of treated sample/OD560 of untreated sample). Assays were performed in triplicate. Standard deviation was calculated from data of obtained from separate experiments.

Transendothelial permeability was determined by transwell experiments. HUVEC cells were grown to confluent monolayer in the upper chamber of the polycarbonate membrane transwell by seeding at 2×10⁶ cells/cm² and incubated for 72 h. Trypan blue-labeled bovine serum albumin was prepared by adding 180 mg trypan blue (sigma) and 4 g BSA fraction V (sigma) to 100 ml HBSS (Gibco) and precipitated with 5% trichloracetic acid. Cultures with or without zVAD-FMK (4 μM) treatment were infected with DENV (MOI=0.1), treated with TNF-α (300 pg/ml), received both DENV and TNF-α or received neither. After incubation for 24 h, 100 μl of trypan blue-stained bovine serum albumin was added to the upper chamber of the transwell at 30 min before the upper chambers were removed. The absorbance of the solution in the lower chamber was measured at 595 nm.

### Western Blot Analysis and Determination of NOx Concentrations and Free Radicals

Primary antibodies rabbit anti-human eNOS (dilution 1:1000, Chemicon, Temecula, Calif.), rabbit anti-human iNOS, (dilution 1:1000) (R&D, Minneapolis, Minn.) or mouse anti-human α-tubulin (dilution 1:1000,Abcam, Cambridge, Mass.) were used. After wash, blots were incubated with a 1/5000 dilution of HRP-conjugated goat anti-mouse or goat anti-rabbit IgG at 4° C. for 1 h. The blots were visualized using the ECL detection system 10 (Amersham Biosciences, Buckinghamshire, United Kingdom) in accordance with the supplier's instructions.

HUVEC culture supernatants were collected at different time intervals after infection with DENV. The Griess reagent kit for nitrite determination (Molecular 16 Probes, Eugene, Oreg.) was used according to the manufacturer's instructions.

2′,7′-dichlorodihydrofluorescein diacetate (DCFH-DA, Molecular Probes) were used to determine endothelial cell production of free radicals. At 45 min after addition of DCFH-DA (5 μcells were washed and resuspended in PBS. Cells were acquired by FACSCanto flow cytometer and analyzed by CellQuest (BD Biosciences) for DCF+_cells.

In vivo and in vitro Treatment with Inhibitors

In vivo treatment with inhibitors was performed as follows. To inhibit the effect of ROS, animals were fed with drinking water containing 40 μg/ml apocynin (Sigma-Aldrich, St. Louis, Mo.) 5 days before DENV inoculation and continued until termination of the experiment. Apocynin was dissolved in absolute ethanol and diluted to1:2000 in sterilized water. The final concentration of ethanol (vehicle) in the drinking water was 0.05%. The drinking water containing apocynin was replenished daily and protected from light. The irreversible, pan-caspase inhibitor Boc-D-FMK (Sigma-Aldrich) and the control peptide Z-FA-FMK (Sigma-Aldrich) were dissolved in DMSO and administered to mice through intraperitoneal injection at 10 μmol/kg. The final concentration of DMSO was 0.05%. Mice were treated daily, beginning at the day of infection until termination of the experiment.

For in vitro treatment with inhibitors, caspase inhibitor benzyl-oxycarbonyl-valyl- alanylaspartic acid fluoro-methyl ketone (zVAD-fmk, 4 μM) (Clontec Laboratories, Palo Alto, Calif.), ceramide inhibitor fumonisin B1 (Cayman, Ann Arbor, Mich.), RNS inhibitor N-nitro-L-arginine methyl ester (L-NAME, 10 nM) (Clontech Laboratories), singly or in combination ROS inhibitor N-acetyl cysteine (NAC, 15 nM) (Sigma-Aldrich) was added to endothelial cell cultures at 30 min before DENV was added and left in the culture throughout the experiment.

### Example 1

### Dengue Hemorrhage is Accompanied by DENV Infecting Endothelium, Macrophage Infiltration, TNF-α Production and Endothelial Cell Death.

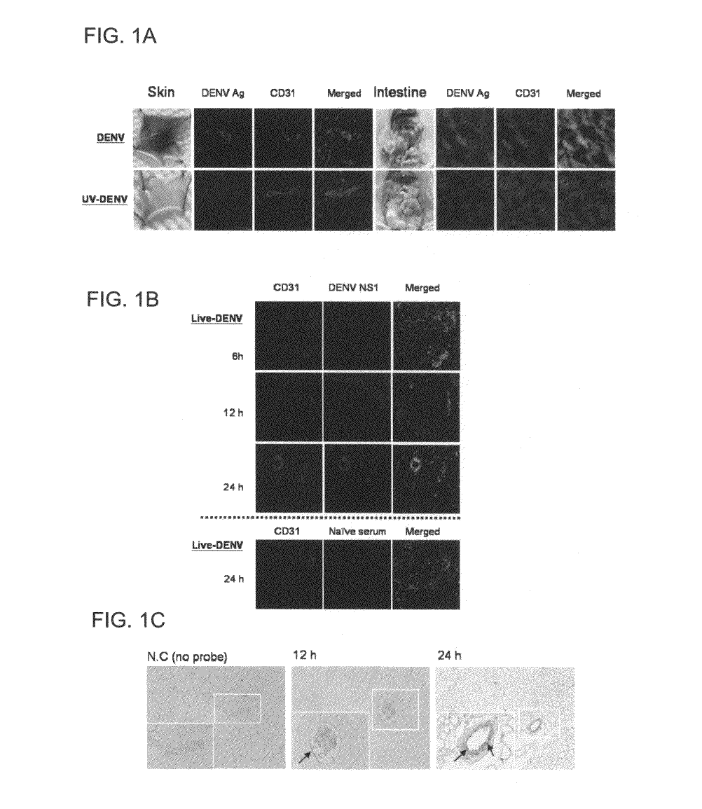

Refer to FIGS. 1A to 1C, which show cryosection images of the hemorrhagic skin and intestine tissues. The data shown are representative of four repeated experiments.

To detect endothelium expression of dengue viral antigen, mice were inoculated intradermally with 2×10⁹ PFU of viable DENV or otherwise equivalent titer of UV-DENV as described above. The skin, subcutaneous and intestinal tissues were harvested at 3 days after infection, and the tissue samples were treated as described above. Cryosections were stained with polyclonal rabbit anti-DENV antibody (kindly provided by Dr. Wen Chang, Institute of Molecular Biology, Academia Sinica, Taipei, Taiwan), plus FITC-conjugated goat anti-rabbit IgG, and PE-conjugated rat anti-mouse CD31 antibody (Invitrogen). Hoechst 33258 was used as a counterstain to stain the nucleic acids. Representative cryosection images of four repeated experiments are shown in FIG. 1B. The original magnification is 630×.

As shown in FIG. 1A, systemic and severe hemorrhage was observed at different anatomic sites after the infection of DENV. Also, immunofluorescence staining showed that CD31⁺ endothelial cells in the vascular endothelium of both the hemorrhagic skin and intestine tissues expressed DENV antigen.

To detect endothelium expression of nonstructural protein 1 (NS1), cryosections of subcutaneous tissues collected from mice intradermally infected with viable DENV (4×10⁷ PFU) at 6, 12 and 24 h or PBS at 24 h after inoculation were stained with mouse anti-DENV NS1 antibody (kindly provided by Dr. Huan-Yao Lei, National Cheng Kung University, Tainan, Taiwan) plus LEXA FLUOR® 594-conjugated goat anti-mouse antibody (Jackson lmmunoresearch, West Grove, Pa.), FITC-conjugated rat anti-mouse CD31 antibody and Hoechst 33258 stain. Naïve mouse serum plus ALEXA FLUOR® 594-conjugated goat anti-mouse antibody and FITC-conjugated rat anti-mouse IgG2a (isotype control) were used as staining controls. Representative cryosection images of four repeated experiments are shown in FIG. 1B. The original magnification is 630×.

Also, skin was harvested from mice intradermally infected with (4×10⁷ PFU) DENV at 12 and 24 h after infection. To detect the viral mRNA, in situ hybridization was performed. In brief, tissue cryosections were first washed in absolute ethanol and dried at room temperature before treatment with proteinase K (100 μg/mL) at 37° C. for 15 min. A biotin-labeled DNA probe (5′CTG-ATT-TCC-AT(ACGT)-CC(AG)-TAX-biotin-3′) at 2 ng/μl was pipetted onto the tissue sections or monolayer before incubation at 45 for 20 hours. Probes that had hybridized with viral RNA were detected by use of horseradish peroxidase (HRP)-conjugated anti-biotin antibody. DAB was used as substrate for color development. The no-probe control was a skin section harvested at 24 h after infection and stained with the biotin detection system without the probe. Representative cryosection images of four repeated experiments are shown in FIG. 1C, where arrows point to viral nucleic acid+endothelial cells. The original magnification is 400×.

Results in FIG. 1B and 1C show that viral NS1 and nucleic acids can be detected at as early as 12 h in the endothelium and continued to increase until 24 h after infection, showing that DENV actively infects and replicates in the endothelium at early phase of the infection.

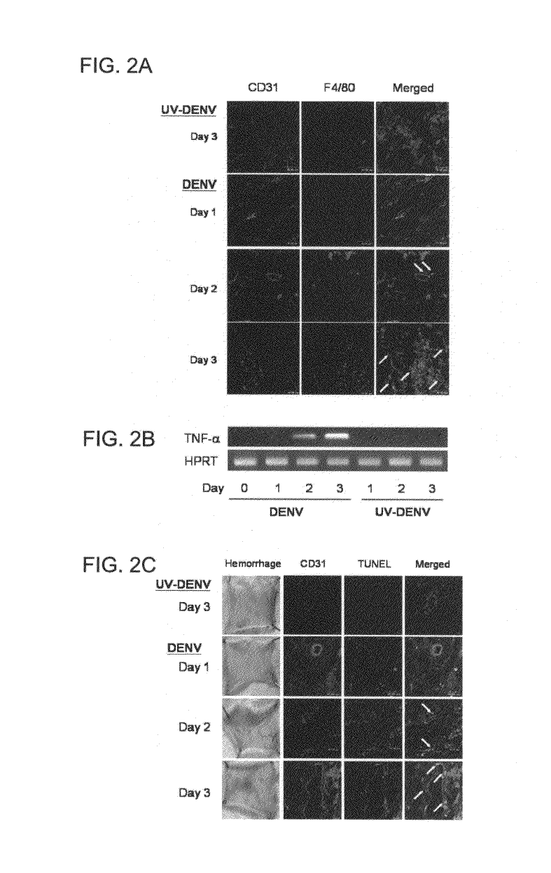

Next, refer to FIGS. 2A to 2C, which show images illustrating that hemorrhage is accompanied by infiltrating macrophages in the vicinity of endothelium, TNF-α production and endothelial cell death. In these images, skins were harvested from mice infected with 2×10⁹ PFU viable DENV or otherwise equivalent titer of UV-inactivated DENV at days 1, 2 and 3 after infection.

To observe the relationship between macrophage and the endothelium, cryosections were stained with both PE-conjugated rat anti-mouse CD31 and FITC-conjugated rat anti-mouse F4/80 (clone BM8, eBioscience) antibodies at 4° C. overnight. Hoechst 33258 stain was used as counterstain. Representative cryosection images of three repeated experiments are shown in FIG. 2A, where white arrows point to F4/80+ macrophages in close proximity to endothelium. Staining with various isotype control antibodies was negative. The original magnification is 630×.

Also, as shown in FIG. 2B, TNF-α mRNA was amplified by RT-PCR, and the result of electrophoresis is shown in FIG. 2B, Housekeeping gene HPRT was used as control.

As shown in FIG. 2C, the skins of infected mice were exposed at days 1, 2, and 3 after infection. The cryosections of the skins were stained with PE-conjugated rat anti-mouse CD31 antibody, FITC-conjugated TdT-mediated dUTP nick-end labeling mixture, and Hoechst 33258 stain. White arrows point to TUNEL⁺CD31⁺ cells. The original magnification is 630×. The data shown are representative 1 of 4 repeated experiments.

Interestingly, after DENV infecting the endothelium, macrophages were observed in the tissues. They appeared as early as day 2 and came into the close proximity of the endothelium by day 3 of infection (FIG. 2A). Coincided with the increase of infiltrating macrophages, the level of TNF-α transcripts also increased (FIG. 2B). TUNEL reaction further revealed that endothelial cells in the hemorrhage tissues became apoptotic at day 2 and 3 after infection, time soon before and when hemorrhage was observed (FIG. 2C).

These kinetics studies demonstrate that hemorrhage development is accompanied by DENV infecting the endothelium macrophage infiltration, TNF-α production and endothelial cells undergoing apoptosis.

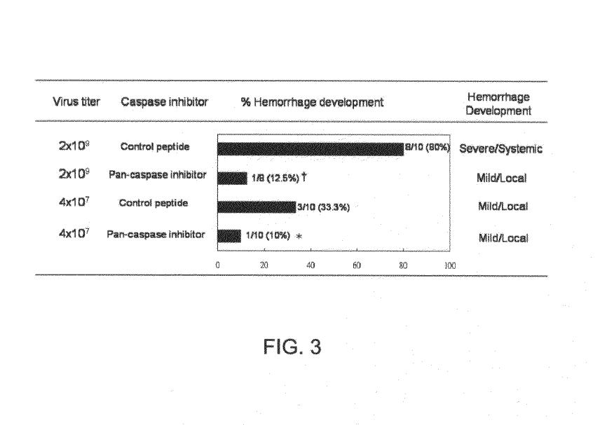

Furthermore, the percentage of mice that developed hemorrhage and the severity of hemorrhage was greatly reduced when mice were treated with pan-caspase inhibitor Boc-D-FMK (FIG. 3), showing the importance of apoptosis, most probably that of endothelial cells, to hemorrhage development.

With respect to the data shown in FIG. 3, mice were inoculated intradermally with the indicated titers of viable DENV. Mice were treated with caspase inhibitor Boc-D-FMK at 10 pmol/kg or equivalent concentration of control peptide Z-FA-FMK daily beginning at the day of DENV infection. Hemorrhage was observed at day 3 after DENV inoculation. +p<0.001 and *p<0.05, comparing mice treated with pan-caspase inhibitor to that treated with control peptide infected with the same titer of DENV.

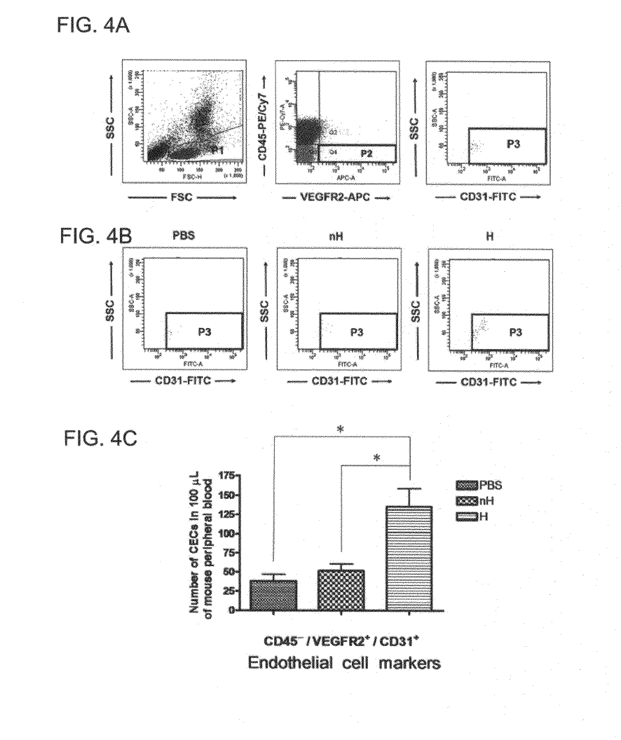

In addition, from mice inoculated intradermally with PBS or 2×10⁹ PFU of DENV, peripheral blood was collected at three days after infection. The percentage of circulating endothelial cells (CEC) was analyzed by flow cytometry as described above. The results are shown in FIGS. 4A to 4B. The data were pooled from three experiments. A total of 100,000 events were acquired for each specimen. *, p<0.05.

FIG. 4A illustrates the strategy for flow cytometric detection of CECs. P1 gate: cells were gated to exclude polymorphonuclear cells, dead cells and debris. P2 gate: CD45⁻VEGFR2⁺ cells were gated to exclude CD45+ leukocytes. P3 gate: VEGFR2⁺CD31⁺ cells were identified as CECs. FIG. 4B shows dot plots illustrating the representative of the gated CECs in uninfected mice (PBS) and from DENV-inoculated mice that did (H) or did not (nH) develop hemorrhage, respectively. FIG. 4C is a bar graph illustrating the numbers of CD45⁻VEGFR2⁺CD31⁺ cells in 100 μl of mouse peripheral blood.

By excluding CD45⁺ leukocytes and gating VEGFR⁺CD31⁺ cells in the peripheral blood, we found that in mice that developed hemorrhage there was significantly greater numbers of circulating endothelial cells (FIGS. 4A-C). These data provide ex vivo evidence to show that vascular damage occurs in the hemorrhage mice and it can be detected by increased number of circulating endothelial cells in the circulation

### Example 2

iNOS Upregulation and Free Radical Production in the Endothelium of Hemorrhage Tissue.

To investigate whether iNOS expression and oxygen radicals are involved in endothelium damage and hemorrhage development, hemorrhage tissues from DENV-infected mice were compared to tissues from mice injected with UV-inactivated DENV.

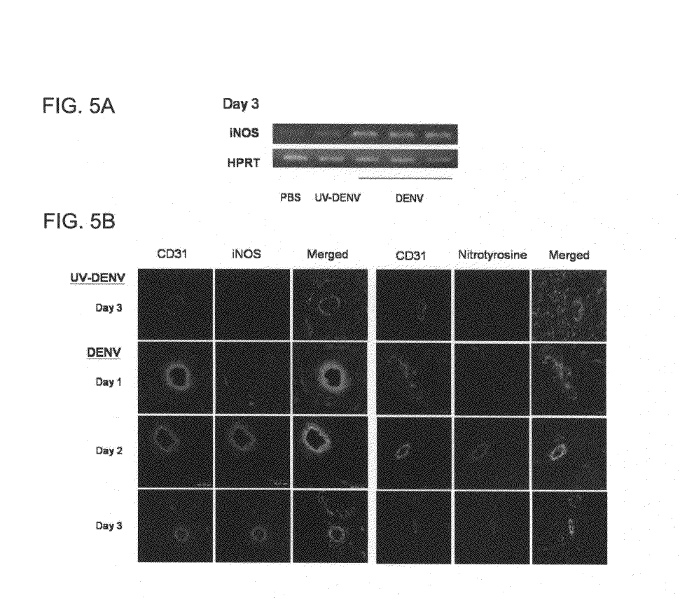

In FIG. 5A, RNA was extracted from subcutaneous tissues of mice receiving PBS, 2×10⁹ PFU of DENV or otherwise equivalent titer of UV-DENV at day 3 after inoculation. iNOS and HPTR mRNAs were amplified by RT-PCR. RT-PCR results show that iNOS mRNA transcripts were up-regulated in the tissues of hemorrhage mice but not in mice injected with UV-DENV (FIG. 5A).

To detect iNOS expression in endothelium, cryosections were stained with PE-conjugated rat anti-mouse CD31 antibody, FITC-conjugated rat anti-mouse iNOS antibody and Hoechst 33258 stain or PE-conjugated rat anti-mouse CD31 antibody, rabbit anti-nitrotyrosine antibody, FITC-conjugated goat anti-rabbit antibody and Hoechst 33258 stain. Immunofluorescence was viewed under confocal microscope. The original magnification is 630×. The data presented in FIG. 5B are representative of 5 repeated experiments.

As can be seen in FIG. 5B, CD31⁺ vascular endothelium in hemorrhage tissues expressed both iNOS and nitrotyrosine beginning at day 2 after infection, demonstrating that vascular endothelium in the hemorrhage tissues produced both high output RNS and ROS, and the temporal kinetics of their production coincided with hemorrhage development. These results strongly suggest that endothelial cell death occurring in the hemorrhage tissues is related to RNS and ROS production.

### Example 3

### Endothelial Cells Support DENV Replication and Undergo Apoptosis After Infection.

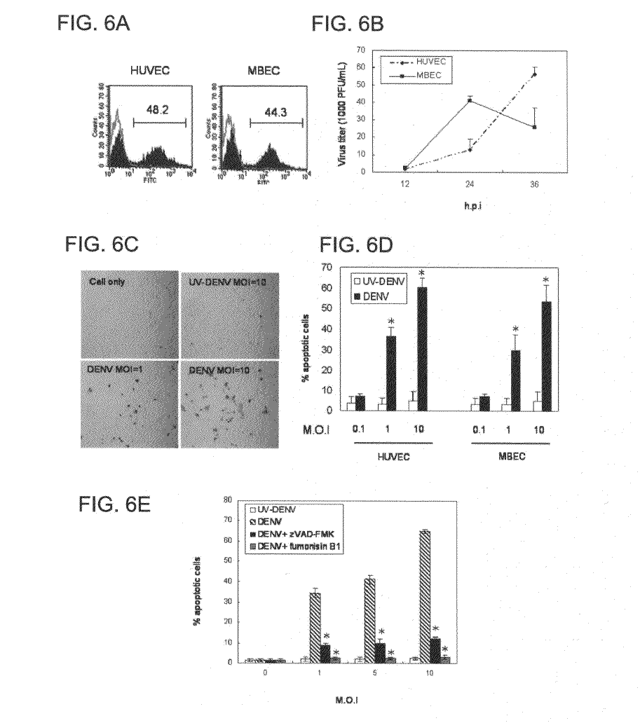

In vitro studies were performed to reveal the relationship between DENV infection of endothelial cells and endothelial cell death. Both MBECs and HUVECs were infected with DENV as described above. Results are shown in FIGS. 6A to 6E.

In FIG. 6A, both HUVECs and MBECs were infected with DENV at MOI of 5. Cells were fixed, permeabilized and stained with rabbit anti-DENV antibody plus FITC-conjugated goat anti-rabbit IgG. Grey line represents uninfected cells and darkened area infected cells. The numbers indicate the percentage of cells stained positive for DENV antigen.

In FIG. 6B, culture supernatants from DENV-infected HUVEC (diamond) and MBEC (square) cells (MOI of 5) were harvested at different time points after infection. Virus titer was determined by plaque assay. Data were pooled from 3 independent experiments and are shown as mean±SD.

The images shown in FIG. 6C illustrate in situ detection of DNA fragmentation where DNA strand breaks were end-labeled with dUTP by terminal deoxynucleotidyl transferase (TdT) using In Situ Cell Death Detection Kit (Roche Applied Science, Indianapolis, Ind.). In FIG. 6C, cells stained brown color are counted as TUNEL+. The original magnification is 200×.

HUVEC were infected with DENV at MOI of 1 or 10 or cultured with UV-DENV at otherwise equivalent of MOI of 10. At 24 h after incubation, cells were harvested and treated as follows. To detect endothelial cell apoptosis in tissues, cryosections were fixed in 4% paraformaldehyde at room temperature for 10 min then treated with 3% H₂O₂ in methanol. Cryosections were then washed and treated with 0.1% Triton®X-100 in 0.1% sodium citrate on ice for 2 min. TUNEL reaction mixture (TdT-mediated dUTP nick end labeling) of enzyme solution (TdT) and label solution (fluorescein-labeled nucleotides) was added and placed at 37° C. for 1.5 h. PE-conjugated rat anti-mouse CD31 antibody was added and left at 4° C. for 1 h. Hoechst 33258 was added after wash. To detect cell death in primary endothelial cell monolayer, converter-POD (peroxidase-conjugated anti-fluorescein antibody) was added and DAB was used as substrate for color development.

The bar graph of FIG. 6D shows the comparisons of percent apoptotic cells in HUVEC and MBEC infected by DENV at different MOI. Percents apoptotic cells were obtained by dividing TUNEL⁺ cells by the total number of cells counted. At least 1000 cells in the each monolayer were counted. Data shown are the mean value±SD of numbers pooled from three independent to experiments. *p<0.05, comparing the % apoptotic cells in DENV-infected monolayer to that in UV-DENV added monolayer.

In FIG. 6E, cells were infected with DENV at MOI of 1, 5 or 10 and cultured in the absence (hatched bar) or presence of 4 μM zVAD-FMK (darkened bar) or 25 μM fumonisin B1 (gray bar) for 30 min before infection and throughout the course of the experiment. Cultures with UV-DENV were used as control (open bar). Percents apoptotic cells were obtained by dividing the numbers of TUNEL⁺ cells by the total number of cells counted. At least 1000 cells in the each monolayer were counted. Data shown are the mean value±SD of percent apoptotic cells pooled from 3 independent experiments. *p<0.05, comparing percent apoptotic cells in zVAD-FMK- or fumonisin B1-treated DENV-infected monolayer to that in control DENV-infected monolayer.

Please refer to FIGS. 6A to 6C. As demonstrated by the presence of intracellular viral protein, 48.2% and 44.3% of HUVEC and MBEC, respectively, were infected by DENV (FIG. 6A). However, while DENV replication in MBEC peaked at 24 h and declined thereafter, that in human endothelial cells continued to rise up to 36 h after infection (FIG. 6B). These results show that both mouse and human endothelial cells supported productive viral infection although the infection kinetics was different in these two types of primary endothelial cells. In the mean time, the endothelial cells became apoptotic and the percentage of apoptotic cells was MOI-dependent (FIGS. 6C and 6D). Moreover, addition of either zVAD-fmk or fumonisin B1, a ceramide inhibitor, significantly reduced DENV-induced cell death, indicating that DENV-induced endothelial cell death is caspase-dependent and involves the activation of de novo ceramide synthesis (FIG. 6E). These results together indicate that endothelial cells of both human and mouse origins support DENV growth, and DENV induces endothelial cell apoptosis.

### Example 4

Endothelial Cells Produce iNOS and Free Radicals in Response to DENV Infection.

To investigate whether DENV-induced apoptosis involves RNS production, HUVEC cells were infected with DENV and iNOS expression was monitored.

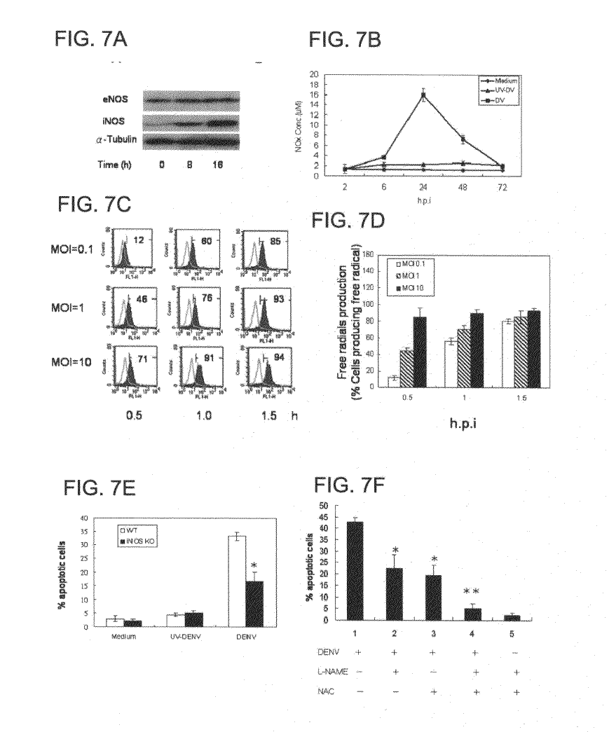

In FIG. 7A, HUVEC was infected by DENV at MOI of 1 and cells were harvested at 0, 8, and 16 h after infection. eNOS, iNOS and α-tubulin expressions were determined by Western blot analysis. The ratios of eNOS/tubulin were 0.8, 0.8, and 0.8, and iNOS/tubulin were 0.1, 0.8, and 1.1, at 0, 8, and 16 h after infection.

Western blot analysis showed that while eNOS was constitutively expressed and not affected by DENV infection, iNOS was induced in infected cells at 8 h after infection and the level of expression continued to increase up to 16 h.

In addition, supernatants from DENV-infected HUVEC at MOI of 1 were collected at different time points after infection and the NOx concentration was determined by Griess assay described above.

As can be seen in FIG. 7B, NOx (mixture of NO⁻, NO2⁻ and NO3⁻) were produced in the culture supernatants of infected cells starting at 6 h after infection and peaked at 24 h.

The results shown in FIGS. 7A and 7B indicate that DENV infection induces endothelial cell iNOS expression and production of high levels of RNS.

To determine whether DENV-induced apoptosis also involves ROS, DENV-infected HUVEC were labeled with DCFH-DA. As DCFH-DA is oxidized by ROS and/or RNS to produce fluorescent DCF, cells expressing DCF are those that can produce ROS and/or RNS.

In FIG. 7C, HUVEC cultures were infected with DENV at MOI of 0.1, 1 or 10 and incubated in the presence of DCFH (5 μM), which is converted by ROS to the fluorescent product DCF, for 30 min. Histograms show DCF fluorescence intensity at 0.5, 1.0 and 1.5 h after DENV infection. The numbers indicate the percentages of cells producing free radicals.

The bar graphs of FIG. 7D show the mean percentage of HUVEC cells producing free radicals at 0.5, 1.0, and 1.5 h after infection by DENV at different MOls. The data shown are the mean value±SD pooled from 4 independent experiments. The data show % free radical-producing cells increased in a time- and MOI-dependent manner.

Results show that free radical-producing endothelial cells increased at MOI- and time-dependent manner (FIGS. 7C and 7D). The results together indicate that DENV infection of endothelial cells induces the production of free radicals.

### Example 5

Blocking both RNS and ROS Completely Reverses DENV-Induced Cell Death.

iNOS-deficient endothelial cells and ROS inhibitors were employed to delineate the involvement of RNS and ROS in DENV-induced endothelial cell apoptosis.

Wild- type (WT) or iNOS−/− murine endothelial cells were infected with DENV or otherwise equivalent titer of UV-inactivated DENV at MOI of 1. At 24 h after infection, the percentage of TUNEl⁺ apoptotic cells was determined by counting 1000 cells per slide. Data shown in FIG. 7E are the mean value±SD pooled from three independent experiments. *p<0.05, comparing MBEC harvested from iNOS−/− mice to that from WT mice.

To observe the effect of the treatment of inhibitor(s) in vitro, cells were pretreated with L-NAME (10 μM), NAC (15 μM) or both for 30 min, and then infected with DENV at MOI of 5. Cells treated with L-NAME and NAC without infection was used as control. At 24 h after incubation, the percentage of TUNEL reaction-positive apoptotic cells was determined by counting 1000 cells per slide. Data shown in FIG. 7F are the mean value±SD pooled from three independent experiments. *p<0.05, **p<0.005, comparing L-NAME- and NAC-treated DENV-infected cells to DENV-infected cells without treatment.

Results in FIG. 7E show that DENV-induced iNOS-deficient endothelial cell death was 17.6±85% at 24 h after infection, ½ of that in the wild-type cells (33.7±3%, p<0.05), demonstrating that RNS is involved, although only partially, in DENV-induced endothelial cell death.

Interestingly, while treatment of endothelial cells with either NAC or L-NAME alone partially reduced DENV-induced endothelial cell death, addition of both inhibitors completely reversed the effect of DENV on the cells (FIG. 7F). Therefore, the effect of dengue virus on endothelial cell death is through the combined activities of RNS and ROS.

### Example 6

TNF-α Enhances DENV-Induced Apoptosis through Increased Production of Both RNS and ROS.

The result of Example 1 presented hereinbefore has shown that TNF-α is critical to hemorrhage development in DENV-infected mice.

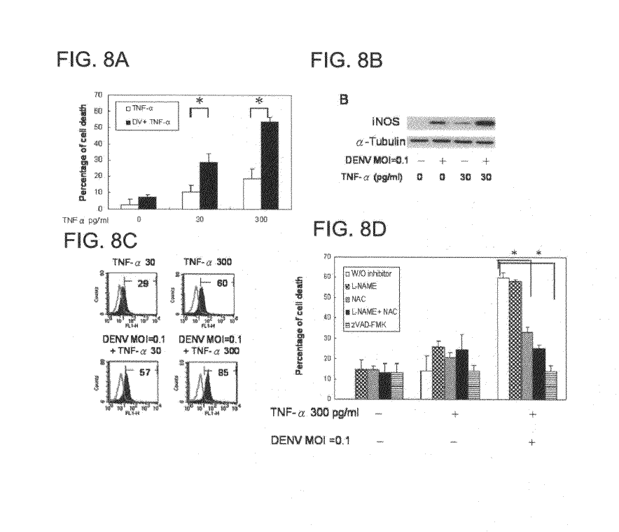

In the present Example, HUVEC cultures with or without TNF-α treatment were infected with DENV at MOI of 0.1. Cell viability was determined by MTT assay at 24 h after infection. From the results shown in FIG. 8A, it is found that TNF-α increased DENV-induced endothelial cell death.

Furthermore, experiments were performed to investigate the effect of TNF-α on endothelial cell production of iNOS and free radicals after DENV infection.

HUVEC was treated with or without TNF-α for 6 h before infection with DENV at MOI of 0.1. Cells were harvested at 24 h after infection and cell lysates were subject to Western blot analysis for iNOS and α-tubulin expressions. The results shown in FIG. 8B are representative of five repeated experiments.

Also, HUVEC cultures were treated with different concentrations of TNF-α for 6 h before infection with DENV at MOI of Cells were incubated in the presence of DCFH (5 μM) for 30 min. Histograms of FIG. 8C show DCF fluorescence intensity, where gray lines represent uninfected control and darkened areas represent DENV-infected HUVEC. The numbers indicate the percentage of cells producing free radicals. The histogram shown is representative of four repeated experiments.

The results of FIGS. 8B and 8C show that while DENV at MOI of 0.1 or TNF-α at 30 or 300 pg/ml alone induced the expressions of low levels of iNOS and free radicals, the presence of both of them greatly increased their expressions, indicating the virus and TNF-α work synergistically in inducing iNOS and free radical production.

In FIG. 8D, HUVEC cultures were pre-treated with L-NAME (10 nM), NAC (15 nM), both L-NAME and NAC, zVAD-FMK (4 μM) or without any inhibitor for 2 h prior to addition of TNF-α (300 pg/ml) and DENV (MOI of 0.1). Cell viability was determined by MTT assay. Data shown are the mean±SD of data pooled from three independent experiments. *p<0.05.

Interestingly, the effect of TNF-α on DENV-induced endothelial cell death was reversed in the presence of NAC alone and the combination of NAC and L-NAME (FIG. 8D). Moreover, zVAD-FMK reversed the effect of TNF-α on DENV-induced cell death (FIG. 8D). These results together indicate that the effect of TNF-α on DENV-induced endothelial cell apoptosis is through enhancing the production of both RNS and ROS, especially ROS.

Refer to the results presented in the Examples hereinabove. It is shown that endothelial cells infected by dengue virus express iNOS and produce RNS and ROS (FIGS. 7A-7D), and endothelial cell apoptosis is suppressed by ceramide synthesis inhibitor, fumonisin B1 (FIG. 6E). In addition, TNF-α enhances DENV-infected endothelial cell iNOS expression and high RNS and ROS production and in the meantime enhances virus-induced apoptosis (FIGS. 8A-8B). Although the inventors do not want to bind this invention with any particular scientific theory, it is speculated that TNF-α synergistically enhances DENV-induced RNS and ROS production through the ceramide-NADPH pathway in the endothelial cells.

Furthermore, addition of NAC and L-NAME reverses the effects of TNF-α and dengue virus on endothelial cells (FIG. 8D). The present application is the first to demonstrate that TNF-α enhances the effect of dengue virus on endothelial cells through augmentation of RNS and ROS productions and such effect is critical to vascular damage in vivo.

Transwell assay was performed to further determine the relations between endothelial cell apoptosis and permeability.

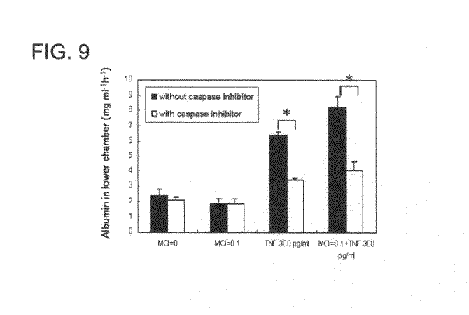

First, HUVEC monolayer in the transwell chamber with and without zVAD-FMK (4 μM) treatment were infected with DENV (MOI=0.1), treated with TNF-α (300 pg/ml), received both DENV and TNF-α or received neither and incubated for 24 h. One hundred pl of trypan blue-stained bovine serum albumin was added to the upper chamber of the transwell at 30 min before the upper chambers were removed. The absorbance of the solution in the lower chamber was measured at 595 nm. In FIG. 9, results were expressed as the amount of BSA detected in the lower chamber. *p<0.05, comparing the group without caspase treatment to that with caspase treatment.

FIG. 9 shows that TNF-α alone and TNF-α plus DENV increased the permeability of endothelial monolayer and addition of zVAD-FMK reduced the effects of TNF-α and TNF-α plus DENV on endothelial cell permeability to the level of controls. These results show that TNF-α- and TNF-α plus DENV-induced endothelial cell apoptosis contributes to the loss of endothelial integrity.

### Example 7

### RNS and ROS are Critical to the Development of Dengue Hemorrhage in Mice.

As we have shown that endothelium in hemorrhage tissues expressed iNOS and nitrotyrosine (FIGS. 5A-B) and that blocking RNS and ROS productions reversed TNF-α- and DENV-induced endothelial cell death in vitro (FIG. 8D), the roles of ROS and RNS in dengue hemorrhage were further investigated in the mouse model.

Wild-type, iNOS^{-/-} or p47^{phox-/-} mice were inoculated with 2 x 10⁹ PFU of DENV intradermally. Separate groups of wild-type and iNOS−/− mice were fed with drinking water containing apocynin (40 mg/ml), a NADPH oxidase inhibitor, starting at 5 days before and continued after intradermal DENV inoculation until termination of the experiment.

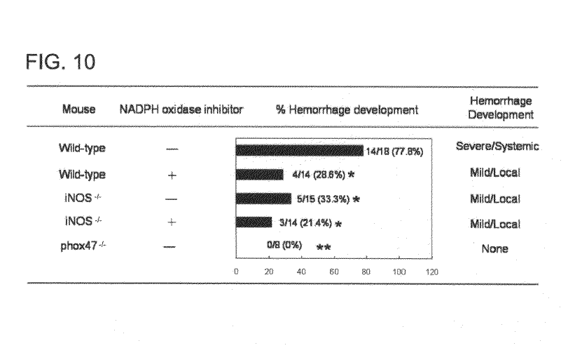

Hemorrhage development was observed when mice were killed at 3 days after DENV inoculation. Percent hemorrhage development was obtained by dividing the number of mice developed hemorrhage with the total number of mice inoculated with DENV. Hemorrhage developed in the skin or subcutaneous tissues on the upper back of the mouse is referred to as mild/local hemorrhage, that developed at multiple anatomical sites is referred to as systemic/systemic hemorrhage. The p value comparing p47^{phox-/-} to wild type mice is **p <0.0001, and that comparing apocynin-treated wild type, iNOS^{-/-}, or iNOS^{-/-} mice treated with apocynin to wild type mice is *p <0.05. The comparison between apocynin-treated wild type mice or iNOS^{-/-} mice treated with apocynin and iNOS^{-/-} mice did not achieve statistical significance. The data were pooled from 2 to 3 experiments. Results are summarized in FIG. 10.

While 77.8% of the wild-type mice infected with DENV developed systemic and severe hemorrhage, 28.6% of wild-type mice treated with apocynin and 33.3% of iNOS^{-/-} mice without apocynin treatment and 21.4% of apocynin-treated iNOS^{-/-} mice developed mild and local hemorrhage (FIG. 10). Interestingly, none of the p47^{phox-/-} mice had hemorrhage manifestations. These results strongly indicate that RNS as well as ROS are involved in DENV-induced hemorrhage in the mouse model and that blocking RNS and ROS production greatly reduces hemorrhage development, pointing to the possibility that antioxidant treatment may be effective in preventing hemorrhage development in dengue virus-infected individuals.

Together with the results presented hereinabove, it is demonstrated that the endothelial cells in the hemorrhage mouse tissue are apoptotic soon before and at the time of hemorrhage development (FIG. 2C), caspase inhibitor reduces permeability change induced by DENV infection and TNFα treatment in vitro (FIG. 9) as well as the percentage and severity of hemorrhage in vivo (FIG. 3). Furthermore, the temporal kinetics of endothelium expressing iNOS and nitrotyrosine corresponds to (FIG. 5B) and oxidase inhibitor, iNOS or 47^{phox} deficiency separately reduces hemorrhage development (FIG. 10). These data together strongly indicate that endothelial cell apoptosis, which contributes to the loss of vascular integrity, is critical to hemorrhage development and that it is mediated by RNS and ROS.

Hence, from the Examples and results presented hereinabove, it is established in temporal sequence that (i) dengue virus infecting the endothelium, (ii) TNF-α stimulating endothelial cells, (iii) endothelial cells expressing iNOS and nitrotyrosine, and (iv) endothelial cells undergoing apoptosis are important events that lead to hemorrhage development.

In summary, by using dengue hemorrhage mouse model and in vitro endothelial cell cultures, the present application demonstrates that dengue virus and TNF-α together induce endothelial cell apoptosis through the production of RNS and ROS. The reaction of RNS and ROS, damaging the endothelium through tyrosine nitration, leads to hemorrhage development. Inhibition of RNS and ROS greatly reduces hemorrhage. The present application reveals the molecular basis for the pathogenesis of dengue hemorrhage and sheds light on potential treatment strategies for dengue hemorrhage.

What is claimed is:

1. A method of treating viral hemorrhagic fever in a subject comprises administering to the subject a therapeutically effective amount of a pharmaceutical composition to reduce the hemorrhage development in the subject, wherein the pharmaceutical composition comprises an oxidase inhibitor and a pharmaceutically acceptable excipient.

2. The method of claim 1, wherein the oxidase inhibitor is selected from the group consisting of apocynin (4-hydroxy-3-methoxyacetophenone), fumonisin B1, diphenyliodonium sulfate, lovastatin, compactin, benzofuranyl- and benzothienyl thioalkane carboxylates, and cytochrome b₅₅₈ fragments and their analogs.

3. The method of claim 1, wherein the subject is a human.


SEQUENCE LISTING (6 sequences)

SEQ ID NO: 1 - DNA, 21 bp, Artificial Sequence
  Chemically synthesized iNOS primer sequence
tgggaatggagactgtcccag

SEQ ID NO: 2 - DNA, 21 bp, Artificial Sequence
  Chemically synthesized iNOS primer sequence
gggatctgaatgtgatgtttg

SEQ ID NO: 3 - DNA, 18 bp, Artificial Sequence
  Chemically synthesized TNF-alpha primer sequence
atccgcgacctcgccctg

SEQ ID NO: 4 - DNA, 19 bp, Artificial Sequence
  Chemically synthesized TNF-alpha primer sequence
accgcctggagttctggaa

SEQ ID NO: 5 - DNA, 25 bp, Artificial Sequence
  Chemically synthesized HPRT primer sequence
gttggatacaggccagactttgttg

SEQ ID NO: 6 - DNA, 22 bp, Artificial Sequence
  Chemically synthesized HPRT primer sequence
gagggtaggctggcctatggct